(19) United States
(12) Patent Application Publication
(10) Pub. No.: US 2006/0178685 A1
(43) Pub. Date: Aug. 10, 2006

(54) Balloon expandable plaque cutting device

(75) Inventor: Jeffry S. Melsheimer, Springville, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/318,989

(22) Filed: Dec. 27, 2005

Related U.S. Application Data
(60) Provisional application No. 60/640,780, filed on Dec. 30, 2004.

(51) Int. Cl. A61B 17/22

(52) U.S. Cl. 606/159; 604/103.05; 604/103.08; 604/22

(57) ABSTRACT

A cutting device for use with a balloon angioplasty catheter of the type having a catheter shaft and an inflatable balloon disposed at a distal portion of the catheter shaft. The cutting device includes a generally cylindrical sleeve sized for mounting on the uninflated balloon. The sleeve is radially expandable upon inflation of the balloon, and includes a plurality of generally curved cut-out portions and raised portions. The raised portions are arranged such that they project radially outwardly from the outer surface of the sleeve, and are sized such that they cuttingly engage plaque deposits encountered upon inflation of the balloon during use of the balloon angioplasty catheter.

### RELATED APPLICATIONS

The present patent document claims the benefit of the filing date under 35 U.S.C. §119(e) of Provisional U.S. Patent Application Ser. No. 60/640,780, filed Dec. 30, 2004, which is hereby incorporated by reference.

### BACKGROUND

1. Technical Field

The present invention relates generally to balloon angioplasty, and more particularly, to a balloon expandable device suitable for cutting plaque in an artery of a patient during a balloon angioplasty procedure.

2. Background Information

Coronary artery disease is a common disease that results in restrictions in the flow of blood to the heart and other areas of the circulatory system. Such restrictions occur primarily due to the formation of obstructions, or stenoses, in one or more of the blood vessels of the patient. The build-up of stenoses, such as plaque, in a blood vessel is a condition referred to in the medical field as atherosclerosis.

A technique that has recently come into widespread use for treating this condition is known as percutaneous transluminal coronary angioplasty (PTCA), or more commonly, balloon angioplasty. Balloon angioplasty is performed to open arteries whose lumens have been restricted due to this build-up of plaque. In a typical balloon angioplasty procedure, an incision is made in a specific area of the patient's body to gain access to an artery, such as the femoral artery. A balloon-tipped catheter is inserted into the artery and threaded through the artery to the site of the blockage. When the blockage site is reached, the balloon is inflated. The inflated balloon pushes the plaque back against the artery wall, thereby clearing the restriction and restoring a pathway for the flow of blood through the vessel. The balloon may be deflated and re-inflated one or more times. If desired, a stent may be inserted at the position of the blockage to prop the artery open.

Balloon angioplasty has been well-received in the medical field as an alternative for the much more invasive and expensive by-pass surgical techniques. Although balloon angioplasty and related procedures have proven successful in many cases for treating coronary artery disease, the procedure is not without its shortcomings. Since the plaque is pressed against the wall of the artery, it is not generally removed from the vessel. In a significant number of patients, the plaque re-forms as a restriction and re-clogs the artery, a condition known as restenosis. When restenosis occurs, it is often necessary to repeat the balloon angioplasty procedure, or, in some instances, undertake more invasive surgical procedures, such as cardiac by-pass surgery. In addition, in some severe cases of stenosis, the plaque deposit may have hardened or become calcified to such an extent that it does not easily yield to the balloon. Use of conventional balloon angioplasty in such instances requires higher pressures, and therefore, requires much care to avoid rupturing the balloon and/or artery at the site of the plaque deposit.

It is desired to provide a device for use in connection with a balloon angioplasty procedure that is capable of breaking down build-ups of plaque from a vessel, and/or of cracking calcified plaque.

### BRIEF SUMMARY

The present invention addresses the problems encountered in the prior art. In one form thereof, the present invention comprises a cutting device for use with a balloon angioplasty catheter of the type having a catheter shaft and an inflatable balloon disposed at a distal portion of the catheter shaft. The cutting device comprises a generally cylindrical sleeve sized for mounting on the balloon when the balloon is in an uninflated condition. At least a portion of the sleeve is radially expandable upon inflation of the balloon. The sleeve includes at least one cut-out portion, and at least one raised portion projecting radially outwardly from an outer surface of the sleeve. The raised portion is sized and shaped to cuttingly engage plaque deposits encountered upon inflation of the balloon during use of the balloon angioplasty catheter.

In another form thereof, the present invention comprises a method for cutting plaque from a body vessel. Initially, an angioplasty catheter assembly is provided. The angioplasty catheter assembly includes a catheter shaft having an inflation lumen, and an inflatable balloon secured to a distal portion of the shaft. An interior portion of the balloon is in fluid communication with the inflation lumen for receiving an inflation fluid therethrough. The assembly further includes a sleeve fitted over an outer circumferential portion of the balloon. At least a portion of the sleeve is radially expandable upon inflation of the balloon. The sleeve includes at least one cut-out portion, and at least one raised portion projecting radially outwardly from an outer surface of the sleeve. The angioplasty catheter assembly is advanced into the vessel with the balloon in an uninflated condition until the uninflated balloon reaches a plaque deposit. The balloon is then inflated such that the sleeve portion radially expands, and the raised portion cuttingly engages the plaque. The balloon is then deflated, and the assembly is removed from the vessel. If desired, the steps of inflating and deflating the balloon can be repeated at least one additional time prior to removal of the assembly from the vessel.

In still another form thereof, the invention comprises an angioplasty catheter assembly for cutting restrictions in a body vessel. The assembly comprises a catheter shaft having an inflation lumen, and an inflatable balloon secured to a distal portion of the shaft. An interior portion of the balloon is in fluid communication with the inflation lumen for receiving an inflation fluid therethrough. A sleeve is fitted over an outer circumferential portion of the balloon. At least a portion of the sleeve is radially expandable upon inflation of the balloon. The sleeve includes a cut-out portion, and a raised cutting portion that projects radially outwardly from an outer surface of the sleeve.

### BRIEF DESCRIPTION OF THE DRAWINGS

FIG. 1 is a perspective view of the distal portion of a balloon angioplasty catheter, illustrating a balloon expandable plaque cutting device of the present invention positioned on the balloon portion of the catheter, with the balloon in an uninflated condition;

FIG. 2 is a side view of the portion of the balloon angioplasty catheter and cutting device of FIG. 1;

FIG. 3 is a sectional view of the balloon angioplasty catheter and cutting device taken along line 3-3 of FIG. 2;

FIG. 4 is an enlarged sectional view of the balloon angioplasty catheter and cutting device taken along line 4-4 of FIG. 2;

FIG. 5 is a side view of the portion of the balloon angioplasty catheter and cutting device shown in FIG. 1, with the balloon in an inflated condition and the cutting device expanded;

FIG. 6 is a side view of the distal portion of a balloon angioplasty catheter, showing another embodiment of an expandable plaque cutting device, with the balloon in an uninflated condition;

FIG. 7 is a sectional view of the balloon angioplasty catheter and cutting device taken along line 7-7 of FIG. 6;

FIG. 8 is an enlarged sectional view of the balloon angioplasty catheter and cutting device taken along line 8-8 of FIG. 6;

FIG. 9 is a side view of the distal portion of a balloon angioplasty catheter, showing still another embodiment of an expandable plaque cutting device, with the balloon in an uninflated condition; and

FIG. 10 is a side view of the portion of a balloon angioplasty catheter and cutting device of FIG. 9, with the balloon in an inflated condition and the cutting device expanded.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the balloon angioplasty catheter, as well as the axial ends of various component features. The term “proximal” is used in its conventional sense to refer to the end of the catheter (or component thereof) that is in closest proximity to the operator during use of the catheter. The term “distal” is used in its conventional sense to refer to the end of the catheter (or component thereof) that is initially inserted into the patient, or that is in closest proximity to the patient during use of the catheter.

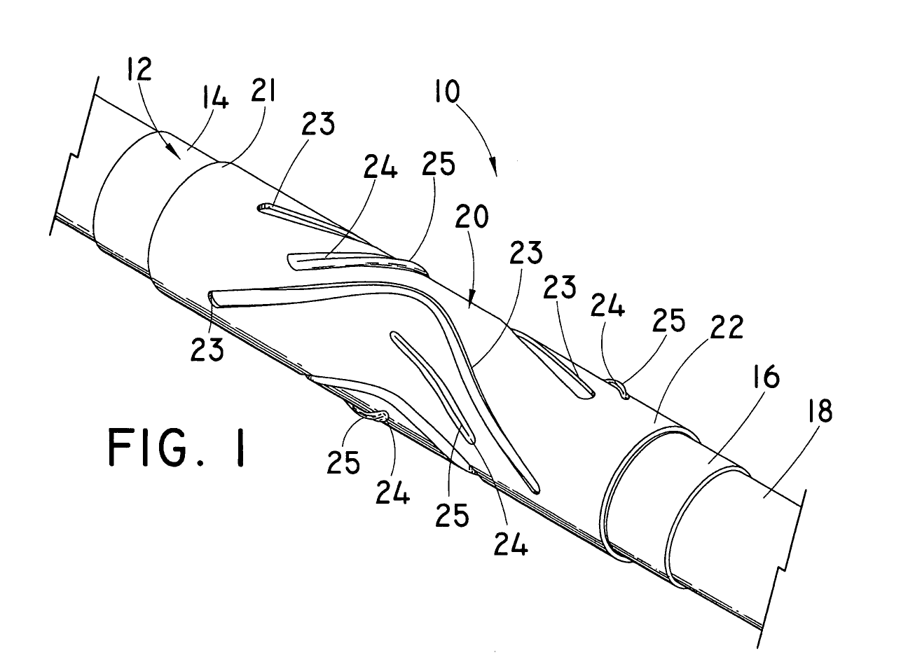

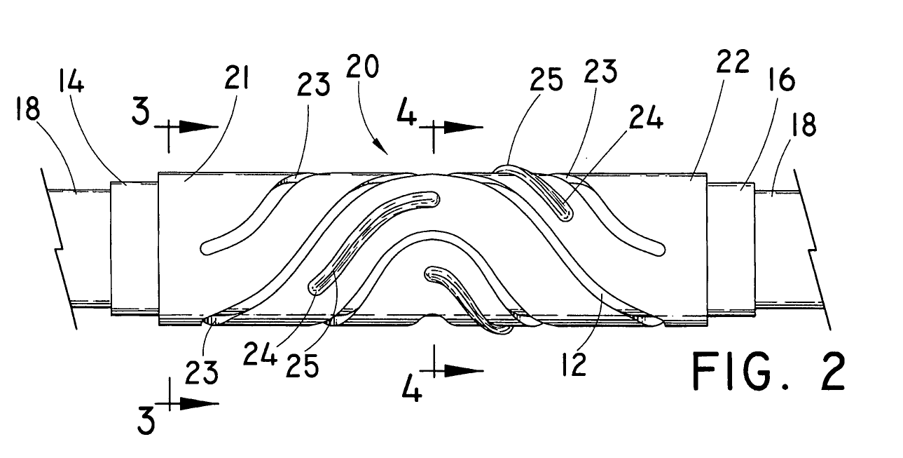

FIG. 1 is a perspective view of the distal, or balloon, portion of a conventional balloon angioplasty catheter 10. FIG. 2 is a side view of the portion of the balloon angioplasty catheter illustrated in FIG. 1. The portion of balloon angioplasty catheter 10 visible in the figures also includes a catheter shaft 18, and an expandable balloon 12 positioned over the distal portion of the catheter shaft in well-known fashion. A balloon expandable plaque cutting device 20, to be discussed hereinafter, is positioned over the balloon. In FIGS. 1 and 2 the balloon is shown in an uninflated condition. Balloon 12 includes a proximal end 14 and a distal end 16. Preferably, balloon 12 is inflatable into a generally cylindrical configuration typical of angioplasty balloons, and comprises a non-compliant balloon of the type commonly used in balloon angioplasty procedures. Non-compliant balloons are well-known for their ability to maintain their inflated diameter under conditions of high pressure, such as the pressures that may be encountered during balloon angioplasty. Other conventional features of a balloon catheter assembly not germane to the present invention have been omitted from the figures and discussion herein.

Balloon expandable plaque cutting device 20 includes a plurality of cut-out portions 23 and raised cutter portions 24. Preferably, raised portions 24 are configured to terminate in a cutting peak 25. In FIGS. 1 and 2, cut-out portions 23 and raised portions 24 are each arranged in a generally C-shaped configuration along the circumference of cutting device 20. Those skilled in the art will appreciate that this arrangement is only one of many possible arrangements of the cut-out portions and raised portions along the circumference of the device, and that other configurations may be substituted. Examples of such alternative configurations include helical configurations, serpentine configurations, as well as various other permutations that can be arranged to provide cut-out portions and raised portions. These portions need not be curved as shown and described, but may instead be provided in more linear-type arrangements, such as in parallel and/or perpendicular configurations along the cutting device. Although any number and shape of cut-out portions 23 and raised portions 24 may be provided, it is generally preferred to have four of each when the generally C-shaped configuration shown and described is utilized.

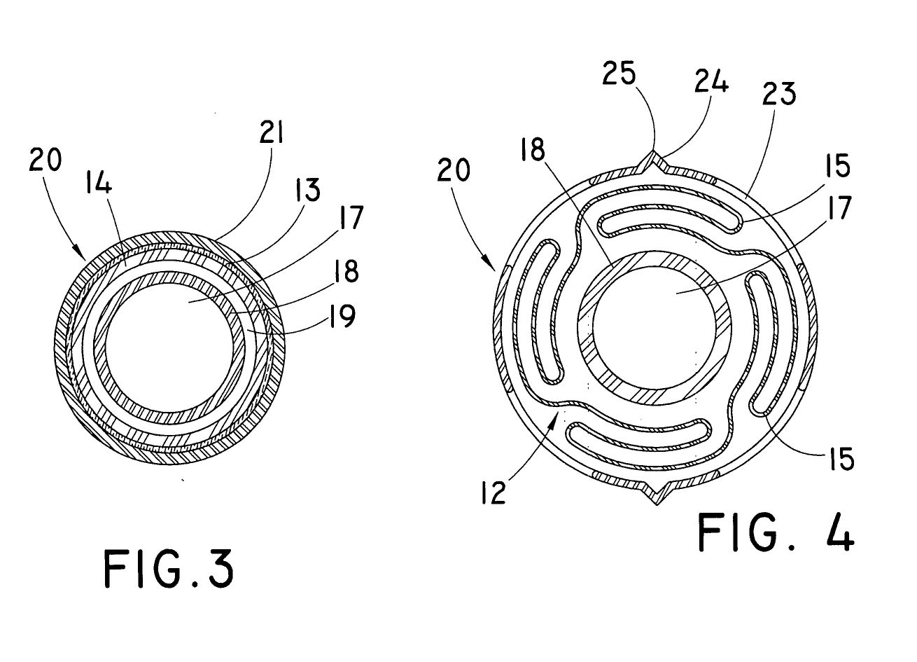

FIG. 3 is a cross-sectional view of the balloon angioplasty catheter and cutting device taken along line 3-3 of FIG. 2. As illustrated, a lumen 17 extends in a longitudinal direction through catheter shaft portion 18 of the angioplasty catheter. Lumen 17 is sized for passage of a wire guide (not shown) therethrough. An inflation lumen 19 is disposed between catheter shaft 18 and proximal balloon portion 14 for selectively transporting an inflation fluid from an inflation source (not shown) to the interior of balloon 12 in conventional fashion to inflate the balloon. In the embodiment shown, a thin layer of adhesive 13 is provided to securely affix the proximal end 21 of expandable cutting device 20 to balloon proximal end 14. Although not visible in the view of FIG. 3, a similar adhesive may be provided to affix cutting device distal end 22 to balloon distal end 16. Those skilled in the art will appreciate that other well-known modes of affixation, such as thermal or chemical bonding, may be utilized in place of an adhesive.

FIG. 4 is a cross-sectional view of the balloon angioplasty catheter 10 and cutting device taken along line 4-4 of FIG. 2. Balloon 12, shown in its uninflated condition, includes a plurality of folded portions 15. In the embodiment shown, the uninflated balloon includes four folded portions 15 substantially equally spaced along the circumference of the balloon. Those skilled in the art will appreciate that other numbers and spacings of folds may be substituted. Upon inflation, folded portions 15 unwrap such that the balloon assumes the conventional cylindrical configuration typical of angioplasty balloons.

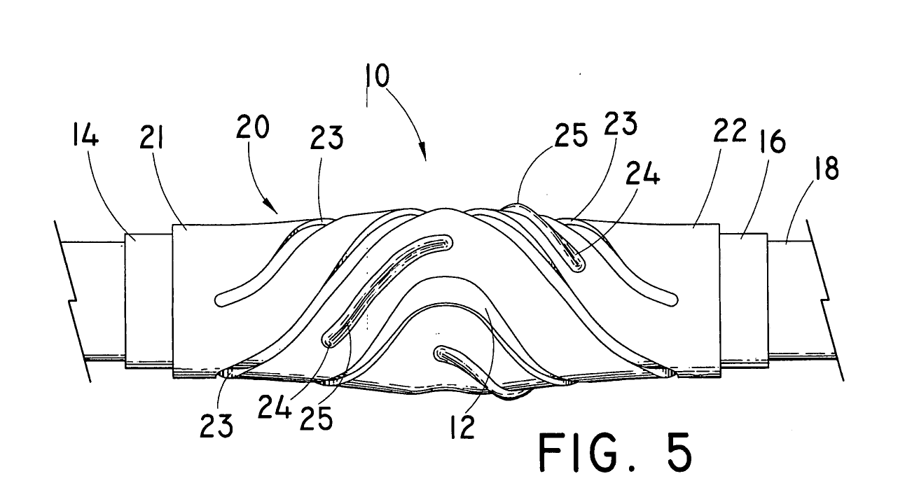

FIG. 5 is a side view of the portion of the balloon angioplasty catheter and cutting device illustrated in FIGS. 1-4, wherein the balloon is in the inflated condition. Upon inflation of the underlying balloon 12, the portion of the cutting device 20 generally intermediate respective proximal and distal ends 21, 22 expands in the radial direction. Cut-out portions 23 function as compliance members to facilitate expansion of the cutting device 20. Following expansion, raised portions 24 are sized and configured such that a peak portion 25 projects radially outwardly for cutting engagement with a stenosis in the body vessel into which balloon angioplasty catheter 10 has been introduced. Peak portions 25 are sized and positioned to cut into the plaque deposits at the blockage site upon inflation of the balloon.

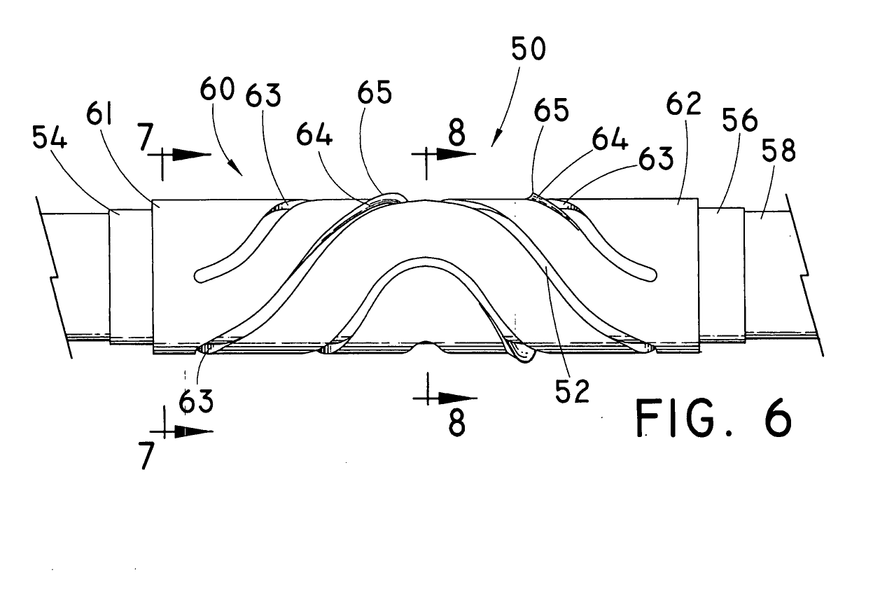

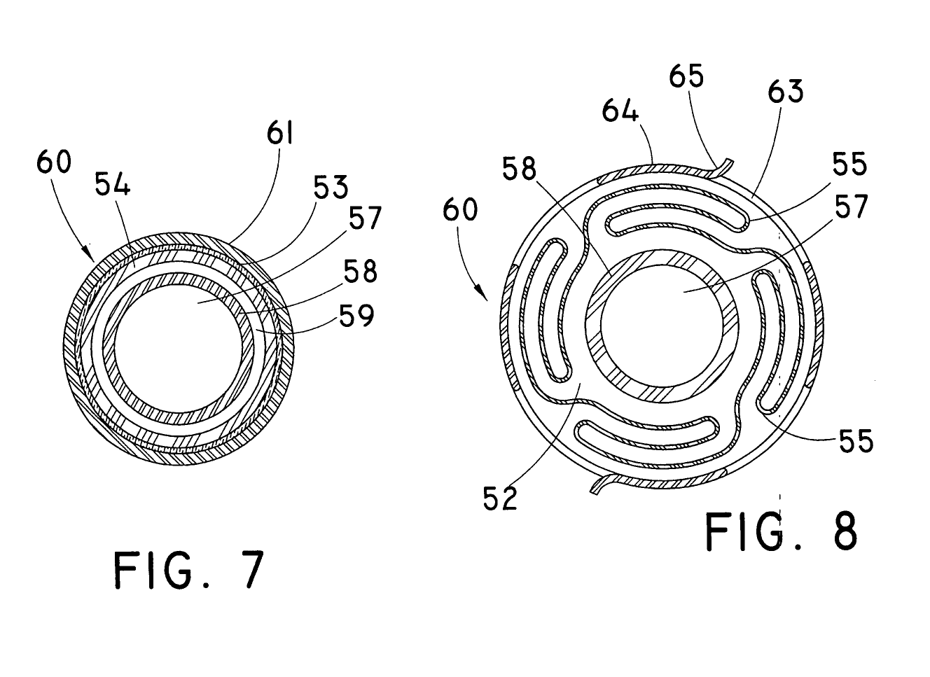

FIGS. 6-8 illustrate a distal portion of another embodiment of a balloon angioplasty catheter 50. In these figures, balloon 52 is shown in an uninflated condition. The uninflated balloon 52 shown in FIG. 6 includes a proximal end 54 and a distal end 56. A balloon expandable plaque cutting device 60 is fitted over the uninflated balloon in the same manner as in the embodiment of FIGS. 1-5. The expandable cutting device, shown in a non-expanded condition in FIG. 6, includes an alternative arrangement of cut-out portions 63 and raised portions 64. In this embodiment, cut-out portions 63 and raised portions 64 are also distributed in a generally C-shaped configuration along the circumference of cutting device 60. Unlike the previous embodiment, however, raised portions 64 are configured to terminate at a cutting peak 65 that is formed on an upturned edge of raised portions 64, rather than being oriented substantially along a center axis. The orientation of cutting peak 65 of this embodiment is best shown in FIG. 8.

In the embodiment of FIGS. 6-8, wire guide lumen 57 and inflation lumen 59 also extend in a longitudinal direction through angioplasty catheter 50, and are separated by angioplasty catheter shaft 58. Adhesive 53 may be provided to securely affix the proximal and distal ends 61, 63 of expandable cutting device 60 to respective balloon proximal and distal ends 54, 56. As shown in FIG. 8, balloon 52 includes folded portions 55 when the balloon is in its uninflated condition.

With the embodiment of FIGS. 1-5, the orientation of raised portions 24 and cutting peaks 25 creates a fulcrum-type effect that assists in breaking or cracking the plaque, rather than solely cutting through the plaque. Thus, this arrangement may be more effective when the obstruction comprises calcified, or near-calcified, plaque. The cutter peak is generally less sharp so that it is less traumatic or invasive. Furthermore, the balloon pressure is generally concentrated directly from the surface of the raised portions 24 to the respective peaks 25.

On the other hand, with the embodiment of FIGS. 6-8, the upturned edge of cutting peak 65 is capable of providing a sharper surface for direct contact with the plaque than in the previous embodiment. The presence of the peak on the outer edge of the raised portion 64 indicates that the cutter may project radially outwardly to a greater extent than the previous embodiment, for a given balloon diameter. Since this design is believed to result in a sharper cutter peak 65 than in the previous embodiment, care must be taken to avoid inadvertent damage to the vasculature, balloon folds, etc., by the sharp edge. In addition, since the cutting peak 65 is located on the outer edge of raised portion 64, the fulcrum effect provided in the previous embodiment is largely diminished or eliminated, thereby lessening the force that may be exerted against the plaque.

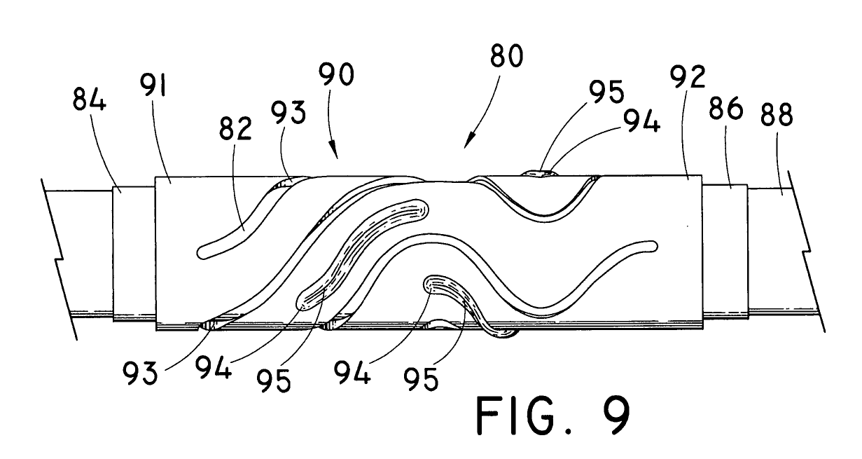

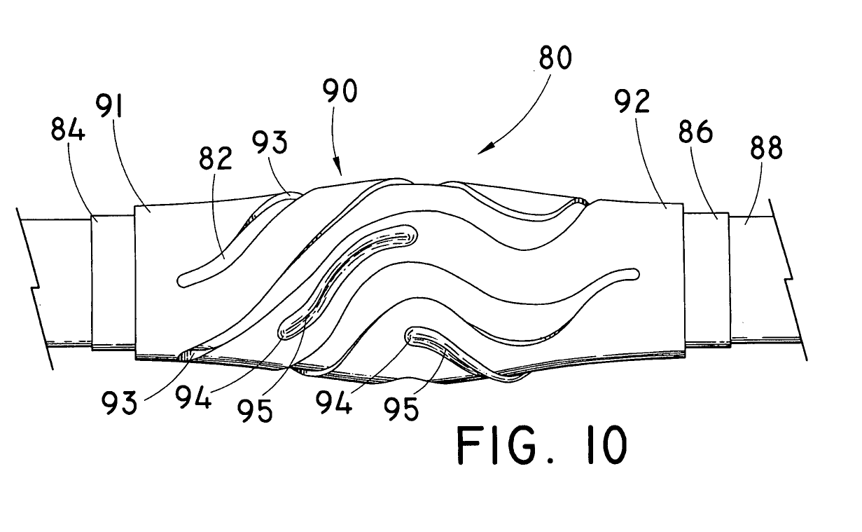

FIGS. 9 and 10 show yet another alternative embodiment of a distal portion of a balloon angioplasty catheter 80. Balloon angioplasty catheter 80 includes a balloon 82 positioned over a catheter shaft 88. In FIG. 9, balloon 82 is shown in an uninflated condition, and in FIG. 10, the balloon is in an inflated condition. Balloon 82 includes a proximal end 84 and a distal end 86. A balloon expandable plaque cutting device 90 is fitted over the balloon. Expandable cutting device 90 illustrates another alternative arrangement of cut-out portions 93 and raised portions 94. In this embodiment, cut-out portions 93 and raised portions 94 are also distributed in a generally S-shaped, or serpentine, configuration along the circumference of cutting device 90. As in the embodiment of FIGS. 1-5, raised portions 94 are configured to terminate at a cutting peak 95, although those skilled in the art will appreciate that the raised portions can alternatively be configured to include an upturned peak, as in the embodiment of FIGS. 6-8, or in another analogous arrangement.

In the view of FIG. 10, balloon 82 is in an inflated condition, and the portion of the cutting device 90 generally intermediate respective proximal and distal ends 91, 92 expands in the radial direction. Once again, cut-out portions 93 are sufficiently compliant to facilitate expansion of the cutting device 90. Following expansion, raised portions 94 are sized and configured such that peak portion 95 engages a stenosis or other obstruction in the body vessel. The presence of the serpentine configuration shown in FIGS. 9 and 10 may be beneficial when it is desired to cut a particularly lengthy plaque deposit, and wherein a more elongated cutting surface may be desired.

Although the embodiments shown include a plurality of cut-out portions and raised portions, the cutting device may include as few as a single cut-out portion and a single raised portion. In this event, in order to optimize the utility of the device, it would be preferred to shape the cut-out portion and/or the raised portion in a winding configuration such that they may cover a large area of the cutting device.

The expandable cutting devices illustrated herein are preferably formed from a cannula having a composition that allows it to expand upon inflation of the underlying balloon, and to return to its original shape upon deflation of the balloon. A preferred composition is a metal or alloy, preferably one having superelastic shape memory properties, such as the nickel-titanium alloy nitinol. Other suitable medical grade shape memory compositions may be used in place of nitinol. Alternatively, other metal or metal alloys having the capability to expand and substantially return to an original shape may also be used. One example of a preferred composition is spring tempered stainless steel. Spring tempered stainless steel has spring-like properties that enable it to expand in the requisite manner for this invention, and thereafter return to its original configuration. Similarly, biologically-compatible composites or polymeric compositions may also be utilized. Those skilled in the art may readily select an appropriate composition in accordance with the teachings of the present invention.

The cut-out portions may be formed in the body of the cutting device in any convenient manner. Those skilled in the art are aware of numerous ways in which cut-outs can be formed in a substrate, and are well able to determine suitable cutting methods without undue experimentation. A particularly preferred method is to laser cut material from the cannula to obtain a desired cut-out configuration.

Similarly, the raised portions may be formed in the body of cutting device in any convenient manner. Those skilled in the art are aware of numerous ways in which raised portions can be formed in a substrate, and are well able to determine suitable methods for forming such portions without undue experimentation. A particularly preferred method is to emboss the raised features from the cannula surface. The cannula can be placed in an appropriate set of male-female dies, and the raised portion can be pushed outwardly in the radial direction while positioned in the die. As another alternative, suitably shaped portions can be affixed to the outside of the cannula. However, in this instance, it is important to ensure that the raised portions are very securely affixed to the cannula, so that they cannot disengage during conditions encountered in the angioplasty procedure.

Those skilled in the art can readily determine appropriate dimensions for the cutting device. The length of the device will generally be dependent upon the length of the underlying balloon. A preferred working length, i.e., a length of cutting portions on the device, is about 5 to 15 mm. The deflated diameter of the device is preferably between about 1 and 1.2 mm, and the inflated diameter is preferably between about 2 and 4 mm. These dimensions are provided as examples only, and are not intended to limit the size of the cutting device in any manner.

As a further variation of the invention, a cutting blade can be inserted in a cutting portion in place of, or as a supplement to, the peaks. The use of a discrete blade can provide a sharper edge, can be formed of any compatible composition, and can be formed to any convenient size and shape. However, the use of a discrete blade adds some complexity to the manufacture of the device, and will require an added element of care during use of the device to insure that inadvertent cuttings are not made to the body vessel, balloon, etc.

Use of the inventive balloon expandable cutting device will now be described. Initially, the device is mounted over the balloon portion of a balloon angioplasty catheter. The balloon angioplasty catheter is inserted into an artery in conventional fashion for a balloon angioplasty procedure. Normally, such insertion is over a wire guide that has been previously been introduced into the artery in conventional fashion, such as via the well-known Seldinger technique. An introducer sheath is inserted into the vessel over the wire guide to establish a pathway from the access site to the site requiring treatment. The catheter may then be directed to a stenosis in the artery, such that the balloon portion is situated in the region of the stenosis. Preferably, the catheter is directed to the treatment site under a conventional medical imaging technique, such as x-ray fluoroscopy.

The balloon may then be inflated by introduction of a suitable inflation fluid through the inflation lumen of the catheter shaft in conventional fashion. Upon inflation of the balloon, the raised portions of the cutting device engage and cut the plaque that comprises the stenosis. If desired, the balloon may be deflated and reinflated one or more additional times. Prior to or during such reinflation, the angioplasty catheter may be rotated such that different portions of the stenosis are cut by the inventive cutting device. The presence of the cut-out portions allows the device to easily expand from its original configuration to the expanded configuration, and return substantially to the original configuration upon deflation of the balloon.

It is therefore intended that the foregoing detailed description be regarded as illustrative rather than limiting, and that it be understood that it is the following claims, including all equivalents, that are intended to define the spirit and scope of this invention.

1. A cutting device for use with a balloon angioplasty catheter of the type having a catheter shaft and an inflatable balloon disposed at a distal portion of the catheter shaft, the cutting device comprising:
a generally cylindrical sleeve sized for mounting on said balloon when said balloon is in an uninflated condition, at least a portion of said sleeve being radially expandable upon inflation of said balloon, said sleeve including at least one cut-out portion, and including at least one raised portion projecting radially outwardly from an outer surface of said sleeve, said at least one raised portion being sized and shaped to cuttingly engage plaque deposits encountered upon inflation of said balloon during use of said balloon angioplasty catheter.

2. The cutting device of claim 1, wherein said sleeve comprises a plurality of cut-out portions and raised portions.

3. The cutting device of claim 2, wherein at least some of said raised portions are arranged in respective C-shaped configurations along the outer surface of the sleeve.

4. The cutting device of claim 2, wherein at least some of said raised portions are arranged in respective serpentine configurations along the outer surface of the sleeve.

5. The cutting device of claim 1, wherein said sleeve comprises a shape memory alloy.

6. The cutting device of claim 1, wherein said sleeve comprises stainless steel.

7. The cutting device of claim 1, wherein said cut-out portions are sized and arranged to facilitate radial expansion of said sleeve during inflation of the balloon.

8. The cutting device of claim 1, wherein said raised portion is configured such that it terminates in a radial direction as a cutting peak.

9. The cutting device of claim 1, wherein said raised portion includes an upturned edge positioned for cutting engagement with said plaque deposits.

10. A method for cutting plaque from a body vessel, comprising:
providing an angioplasty catheter assembly, said angioplasty catheter assembly comprising a catheter shaft having an inflation lumen, and an inflatable balloon secured to a distal portion of the shaft, an interior portion of said balloon in fluid communication with said inflation lumen for receiving an inflation fluid therethrough, said assembly further comprising a sleeve fitted over an outer circumferential portion of the balloon, at least a portion of said sleeve being radially expandable upon inflation of said balloon, said sleeve including at least one cut-out portion, and at least one raised portion projecting radially outwardly from an outer surface of said sleeve;
advancing the angioplasty catheter assembly with said balloon in an uninflated condition into the vessel until said uninflated balloon reaches a plaque deposit; and
inflating said balloon such that said sleeve portion radially expands and said at least one raised portion cuttingly engages said plaque.

11. The method of claim 10, further comprising the steps of deflating the balloon, and repeating the inflating and deflating steps at least one additional time.

12. The method of claim 11, further comprising the step of rotating the catheter shaft prior to repeating an inflation step.

13. The method of claim 11, wherein said angioplasty catheter assembly is advanced into said vessel utilizing the Seldinger technique.

14. The method of claim 10, wherein said sleeve comprises a plurality of cut-out portions and raised portions.

15. The method of claim 14, wherein said raised portions are arranged in respective curved configurations along the outer surface of the sleeve.

16. The method of claim 10, wherein said sleeve comprises a member selected from the group consisting of shape memory alloys and stainless steel.

17. An angioplasty catheter assembly for cutting restrictions in a body vessel, said assembly comprising:
a catheter shaft having an inflation lumen, and an inflatable balloon secured to a distal portion of the shaft, an interior portion of said balloon in fluid communication with said inflation lumen for receiving an inflation fluid therethrough; and
a sleeve fitted over an outer circumferential portion of the balloon, at least a portion of said sleeve being radially expandable upon inflation of said balloon, said sleeve including a cut-out portion, and a raised portion projecting radially outwardly from an outer surface of said sleeve.

18. The assembly of claim 17, wherein a proximal end of said sleeve is affixed to a proximal end portion of said balloon, and a distal end of said sleeve is affixed to a distal end portion of said balloon.

19. The assembly of claim 17, wherein said sleeve comprises a plurality of cut-out portions and raised portions, at least some of said cut-out portions and raised portions having a generally curved configuration.

20. The assembly of claim 19, wherein said cut-out portions are sized and arranged to facilitate radial expansion of said sleeve during inflation of the balloon.
